(19) United States
(12) Patent Application Publication
(10) Pub. No.: US 2020/0309798 A1
(43) Pub. Date: Oct. 1, 2020

(54) METHOD FOR DETERMINING CARDIOVASCULAR RISK BY POSTPRANDIAL CHANGES IN HDL3

(71) Applicant: LIPIDRISK, LLC, The Woodlands, TX (US)

(72) Inventor: Jan M. TROUP, The Woodlands, TX (US)

(21) Appl. No.: 16/761,029

(22) PCT Filed: Nov. 2, 2018
(86) PCT No.: PCT/US18/59055

§ 371 Date: May 1, 2020

(30) Foreign Application Priority Data
Nov. 3, 2017 (US) 62581594

(51) Int. Cl. G01N 33/92

(52) U.S. Cl. CPC G01N 33/92; G01N 2800/323

(57) ABSTRACT

Embodiments of the present invention include a method (10) for assessing a patient for cardiovascular risk. The method includes the steps of: drawing a first blood sample from a patient in a fasting condition; administering a liquid based protein meal to the patient; drawing a second blood sample from the patient after a time period of 60-120 minutes after the step of administering the meal; measuring HDL3-P of the first blood sample so as to determine a fasting HDL3-P amount (20); measuring HDL3-P of the second blood sample so as to determine a postprandial HDL3-P amount (30); comparing the fasting HDL3-P amount and the postprandial HDL3-P amount; and detecting cardiovascular risk based on the comparison.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to detection of cardiovascular disease in mammals. More particularly, the present invention relates to detection of cardiovascular disease in humans by an assay of postprandial changes in particle numbers for the lipoprotein subgroup HDL3. The present invention also relates to adjusting subsequent activity and treatment, after detecting cardiovascular disease by the method.

2. Description of Related Art Including Information Disclosed Under 37 CFR 1.97 and 37 CFR 1.98

Cardiovascular disease is a condition affecting the heart and blood vessels. Cardiovascular disease causes numerous health problems, which tend to be related to plaque deposits narrowing arteries in a condition known as atherosclerosis. A heart attack, ischemic stroke, hemorrhagic stroke, heart failure, arrhythmia, and heart valve problems are all risks of cardiovascular disease. Detection of cardiovascular disease allows for intervention and treatment so that a patient may reduce the risks of serious health consequences.

High Density Lipoprotein (HDL) particles have different subgroups, distinguished by density and size. HDL particles are considered to be the “good cholesterol” because HDL particles can remove fat molecules from cells and the arterial intima. Excess fat molecules, such as cholesterol, phospholipids, and triglycerides, are removed to reduce the risk of atherosclerosis as a normal body process. HDL particles are primarily comprised of cholesterol, phospholipids, and the protein apolipoprotein A1 (Apo A-1). Elevated concentrations of HDL particles are strongly associated removing plaque deposits (fat molecules) from artery walls to reverse atherosclerosis.

The HDL3 particle types are the smallest and most dense of the HDL particles and are made predominately in the liver and intestines as small spherical particles. These HDL3 particles circulate throughout the body going in and out of cells picking up free cholesterol and, to a lesser extent, cholesterol that has been esterified by the enzyme Lecithin-cholesterol acyltransferase (LCAT). As the HDL3 particles pick up more and more cholesterol they become medium size spherical particles called HDL2 particles, starting initially as medium size HDL2a particles. The end result of this process from HDL3 particles to HDL2 particles is the formation of the largest and most buoyant HDL2b particle which is loaded with cholesterol. This mature HDL2b particle goes back to the liver to drop off the cholesterol stores in the process called reverse cholesterol transport (RCT). The prior art supported the HDL2b particle as the best indicator of HDL functionality; and high amounts of the HDL2b particles were found to be correlated with reduced cardiovascular risk.

Further investigation of the reverse cholesterol transport (RCT) indicated that the HDL3 particles may also be used as an indicator of HDL functionality and may also be related to cardiovascular risk. See Oestreicher Stock et al., Levels of prebeta-1 high-density lipoprotein are elevated in 3 phenotypes of dyslipidemia: Journal of Clinical Lipidology (2018) 12, 99-109. See also Gillard et al., Rethinking reverse cholesterol transport and dysfunctional high-density lipoproteins. J. Clinical Lipidology (2018) 12, 849-856. The results showed that HDL3 particles gather free cholesterol and returns the free cholesterol to the liver as the main reverse cholesterol transport path. The HDL2b particles were still also confirmed in the reverse cholesterol transport (RCT) process; however, the RCT process pathway for the HDL3 particles had a half-life of 7-8 minutes, while the RCT process pathway for the HDL2b particles had a half-life of 0.92 days. The RCT process pathway for the HDL3 particles appeared to be the main pathway for returning cholesterol to the liver, while the HDL2b appeared to be a secondary pathway.

Additionally, HDL3 particles are also known to be powerful antioxidant and anti-inflammatory agents from the association with paroxyonase-1 (PON1), which protects LDL particles from oxidation. Oxidized LDL particles are a harmful type of cholesterol, and HDL3 particles can prevent the oxidation of LDL particles. See Handrean Soran, Jonathan Schofield and Paul N. Durrington, Frontiers in Pharmacology (2005) 6:222. Additionally, the HDL3 particles appear to be a more powerful antioxidant than the HDL2 particles. See Kontush A, Chapman M J, Nat Clin Pract Cardiovasc Med. (2006) March; 3(3):144-53. In particular, the HDL3 particles may protect the LDL from oxidation by reducing lipid hydroperoxides to hydroxides. See Brites F, Martin M, Guillas I, Kontush A, BBA Clinical 8 (2017) 66-77. See also Zerrad-Saadii et al., Arterioscler Thromb Vasc Biol. (2009)29:2169-2175.

These properties of the HDL3 particles were previously considered an afterthought in the treatment of cardiovascular disease. The prior art understanding on the HDL2b particles remains correct and concurrent with the further investigation of the HDL3 particles. Even with HDL3 particles as a “main pathway” for the reverse cholesterol transport (RCT) process, the HDL2b particles can still be an indicator of cardiovascular health as a “secondary pathway”. Although different from the HDL2b particles, the HDL3 particles did appear to have some relation to cardiovascular health also. The antioxidant and anti-inflammatory properties were just possible side benefits. In the prior art, there is little information, understanding, and confirmation for how HDL3 can be used as an activated assay or as a potential target for a therapeutic agent.

It is now possible to separately measure HDL3 particles, including ultracentrifugation, ion mobility, dimensional electrophoresis, and NMR technology. See Table 1 in FIG. 1.

HDL3 particles are a combination of different types of HDL3 particles: HDL3a, 3b and 3c. The subgroups of HDL3 particles can be defined and separated by density using ultracentrifugation or by size using gel electrophoresis and measured as a percentage of total HDL (Table 1). Ultracentrifugation can either measure HDL cholesterol such as in the Vertical Auto Profile (VAP) method or HDL particle numbers as in the Lipoprotein Particle Profile (LPP (registered trademark)). Ion Mobility separates HDL by size and reports particle numbers as large, 10.5-14.5 nm equal to HDL2b or small 7.6-10.5 nm equal to HDL2a plus HDL3 with results having been reported with a high coefficient of variation. 2-Dimensional electrophoresis measures the content of Apo A-1 protein in subgroups named pre?-1 and ?-1 to ?-4 with ?-4 plus ?-3 having sizes of 7.4 to 8.1 nm, approximately HDL3, ?-2 size of 9.2 nm, approximately HDL2a and ?-1 with a size of 11.0 nm approximately that of HDL2b. In this technology the Apo A-1 protein content can vary from 1 to 5 Apo A-1 molecules per particle making a particle number determination difficult. Another measurement of HDL particle numbers is the NMR technology. This technology does not separate the particles but claims to measure HDL particle numbers through a NMR signal de-convolution routine. HDL particles are reported as small, medium and large, presumably equal to HDL3 and HDL2 with recent work stating that small+medium HDL particles are a proxy for HDL3-C and large HDL particles are highly correlated to HDL2-C.

Until recently HDL3 cholesterol (HDL3-C) or HDL3 particles (HDL3-P) levels have been thought to be unimportant as a risk indicator other than contributing to the total HDL particle number. Small HDL has been noted in a study as being low in at least one disease state called Apo C-1 enriched HDL. See Kwiterovich, P. O. et al., A Large High-Density Lipoprotein Enriched in Apolipoprotein C-I, JAMA, Apr. 20, 2005-Vol 293, No. 15. In this study, low small HDL, low VLDL and the presence of very buoyant HDL2b, sometimes showing a peak at a density of about 1.060 g/ml, contained a high level of Apo C-1; and the Apo C-1 was thought to be the atherogenic component. Later work showed that the Apo C-1 enriched HDL is a pro-inflammatory HDL. See Catherine J. McNeal et al., Human HDL containing a novel apoC-I isoform induces smooth muscle cell apoptosis, Cardiovascular Research (2013) 98, 83-93. In patients with cardiovascular disease, showing the profile characteristic of low VLDL, low small HDL and high and very buoyant HDL2b, the protein Apo C-1 was determined to have a 90 Dalton mutation. See D'Vesharronne Moore, Catherine McNeal, Ronald Macfarlane, Isoforms of apolipoprotein C-I associated with individuals with coronary artery disease, Biochemical and Biophysical Research Communications 404 (2011) 1034-1038). Apo C-1 is known to activate LCAT, thereby esterifying more cholesterol and increasing HDL2b and it also activates lipoprotein lipase (LPL) which converts VLDL particles to LDL particles giving the characteristics of the observed atherogenic profile. See N. Rifiai, G. Warnick, M. Dominiczak (Eds.), Handbook of Lipoprotein Testing, AACC Press, Washington, D.C., 1997. It is also thought that Apo C-1 may inhibit CETP which would increase HDL. In these studies, the prospect that lower, small HDL may have contributed to the atherogenic nature of the Apo C-1 enriched HDL was not discussed. This form of HDL is observed in 1-2% of the population. The smaller amounts of the smaller HDL3 particles appear related to contributing to atherosclerosis.

The smaller HDL3 appears primarily responsible for the inverse association of HDL-C and cardiovascular disease, as HDL3 particle types have been further studied and investigated.

Several of recent studies have been published measuring cholesterol in HDL subgroups by ultracentrifugation separation or detergent based separation and one measuring HDL particle numbers using NMR. A study looking at LDL and HDL subgroups in the AIM-HIGH clinical trial found that HDL3 cholesterol (HDL3-C) may be primarily responsible for the inverse association of HDL-C and cardiovascular disease. See John J. Albers, April Slee, Jerome L. Fleg, Kevin D. O'Brien, Santica M. Marcovina, Relationship of baseline HDL subclasses, small dense LDL and LDL triglyceride to cardiovascular events in the AIM-HIGH clinical trial, Atherosclerosis 251 (2016) 454-459. The authors explain that “one explanation for this finding could be that this denser HDL3-C subclass containing smaller HDL particles may better reflect the cholesterol efflux capacity and antioxidant, anti-inflammatory, anti-thrombotic, and anti-apoptotic properties of small, dense HDL particles than does HDL2-C”. The study was performed using a detergent-based HDL3-C assay developed by Denka Seiken, Tokyo, Japan. The hazard ratio for the lowest quartile of HDL3-C was significantly higher after 5 years.

Two studies have been done using the ultracentrifugation method (Vertical Auto Profile—VAP) to separate HDL3 and measure the cholesterol content. The study by Joshi et al., Association of high-density lipoprotein subclasses and incident coronary heart disease: The Jackson Heart and Framingham Offspring Cohort Studies, European Journal of Preventive Cardiology 2016, Vol. 23(1) 41-49. looked at the Jackson Heart Study and Framingham Offspring Cohort and measured total HDL-C, HDL2-C and HDL3-C. This study concluded that “smaller, denser HDL3-C levels are primarily responsible for the inverse association between HDL-C and incident CHD in this diverse group of primary prevention subjects”. They also drew the conclusion that “The biological complexities of the reverse cholesterol transport system suggest that a simple “snapshot” of cholesterol content within HDL species at a single time point may be an inadequate surrogate for gauging reverse cholesterol transport efficiency.

In a similar study, identified in Martin et al., HDL cholesterol subclasses, myocardial Infarction, and mortality in secondary prevention: the lipoprotein investigators collaborative, European Heart Journal (2015) 36, 22-30, the authors looked at the TRIUMPH and IHCS studies for secondary prevention of myocardial infarction and mortality. The conclusion of this study was “[I]n secondary prevention, increased risk for long-term hard clinical events is associated with low HDL3-C, but not HDL2-C or HDL-C, highlighting the potential value of subclassifying HDL-C”. They also concluded that “HDL3 particles play a central role in RCT, extracting cholesterol from the periphery, and mature into HDL2 particles via progressive lipidation by lecithin:cholesteryl acyltransferase. Indeed, apolipoprotein A1 (Apo A-1) is considered the major structural HDL protein and is more tightly correlated with HDL3-C than HDL2-C in our data”. However, these findings are disputed by an in-house study showing the exact opposite, i.e., that Apo A-1 is highly correlated with HDL2b and to a lesser extent to HDL2a and that HDL3 is slightly negatively correlated with Apo A-1. Martin et al. continues with “The central positioning of HDL3 in RCT and greater contribution to total HDL-C may indicate that HDL3 assumes the majority of responsibility for HDL functions, such as RCT.

An NMR particle number study looking at carotid intima media thickening was performed using data from the MESA study. See Kim D. S. et al., Concentration of Smaller High-Density Lipoprotein Particle (HDL-P) Is Inversely Correlated With Carotid Intima Media Thickening After Confounder Adjustment: The Multi Ethnic Study of Atherosclerosis (MESA), J Am Heart Assoc. 2016; 5:e002977. In this study, the authors used the measurement of small plus medium HDL particles as a surrogate for HDL3-C. The concluding summary was “we have performed analyses on participants in the large, well-characterized MESA cohort and report that small+medium HDL-P is strongly and inversely associated with cIMT in both the common and internal carotid arteries. Large HDL-P is not associated with cIMT in either carotid artery branch after adjustment for HDL-C, suggesting that cholesterol carried by HDL is likely not important in prevention of cardiovascular and cerebrovascular disease”.

These prior studies represent the established understanding and utility of HDL3 particle types. Elevated HDL2-C cholesterol may represent a saturated system that is not being efficiently offloaded and is limited in further uptake of peripheral cholesterol” and additionally said that “measures of HDL functionality are not clinically practical currently”. The results of the combined studies gave a hazard ratio of 0.77, p=0.007 for HDL3-C and 0.92, p=0.35 for HDL2-C. Additionally, the results show that the hazard ratios for both the TRIUMPH and the IHCS studies were significantly higher for the lowest HDL3-C tertile with a three years ratio of 1.80 and a five year ratio of 1.55. The results also show that although the small+medium HDL's are called a proxy for small HDL-C, it still is the only study of the measurement of small+medium HDL particle number to cardiovascular development and risk. The prior studies looked at both common and internal cIMT thicknesses to assess cardiovascular disease development as a function of small+medium HDP-P by quartile and found a significant increase in the lowest quartile. This result once again emphasizes the value of the low tertile or quartile of HDL3, either as particles or cholesterol, in cardiovascular risk assessment.

Additional prior art supports as reverse finding about the HDL3 particles and cardiovascular disease. The relationship of HDL3 particles is not so clearly an inverse association of HDL-C and cardiovascular disease. High levels of HDL3 are also associated with cardiovascular disease.

The Veterans Affairs HDL Intervention Trial, VA-HIT Study using 2-D Electrophoresis shows a direct association with cardiovascular disease. See Asztalos et al., Value of High-Density Lipoprotein (HDL) Subpopulations in Predicting Recurrent Cardiovascular Events in the Veterans Affairs HDL Intervention Trial (VA-HIT): Arterioscler Thromb Vasc Biol. 2005; 25:2185-2191. The Postmenopausal Women Study also shows a direct association with cardiovascular disease, using 2-D Electrophoresis. See Lamon-Fava et al., Plasma Levels of HDL Subpopulations and Remnant Lipoproteins Predict the Extent of Angiographically-Defined Coronary Artery Disease in Postmenopausal Women: Arterioscler Thromb Vasc Biol. 2008; 28:575-579, arrived at the same conclusion.

With the prior art split, there is a need to reconcile the results from both ends of the inverse or direct relationship to cardiovascular disease.

From the earlier mentioned study by Oestreicher Stock et al. (See Oestreicher Stock et al., Levels of prebeta-1 high-density lipoprotein are elevated in 3 phenotypes of dyslipidemia: Journal of Clinical Lipidology (2018) 12, 99-109), elevated pre-Beta-1 HDL, the precursor of HDL3c, shows significant evidence for high risk in high LDL-C, combined hyperlipidemia and hypertriglyceridemia patients. This work is in agreement with the observation that HDL3-P is elevated and HDL2b-P is low in metabolic syndrome patients. In healthy individuals with low fasting triacylglycerols and high HDL2b-P, the HDL3-P is usually low, which is also in agreement with other studies. The results are shown in Table 2 of FIG. 2.

Healthy individuals usually have low fasting triacylglycerols, high HDL2b-P, and low HDL3-P. However, the Metabolic Syndrome patients usually have high fasting triacylglycerols, low HDL2b-P values, and high HDL3-P. The levels of triacylglycerols differentiate the healthy individuals and the metabolic syndrome patients. The metabolic syndrome patients are individuals experiencing high blood pressure, high blood sugar, excess body fat around the waist, or abnormal cholesterol levels. That is, the metabolic syndrome patients have some level of dysfunctional reverse cholesterol transport already.

Another known study is the impact of meal composition on postprandial lipid and lipoprotein particle numbers using both high protein (HP) and high mono-unsaturated fat (HMF) diets. See Meena Shah et al., Effect of meal composition on postprandial lipid concentrations and lipoprotein particle numbers: A randomized cross-over study, PLOS ONE|DOI:10.1371/journal.pone.0172732 Feb. 21, 2017. In this study it was noted that postprandial total HDL-P and HDL-P-HDL2b-P (medium and small HDL particles) increased with both high protein and high mono-unsaturated fat diets at 120 and 180 minutes. The high protein diet increased these values significantly more than the high mono-unsaturated fat diet, and the increases were not significantly different between the 120 and 180 minute postprandial times. The increase in total HDL-P was approximately the same as the medium and small HDL particle increase so the effect can be attributed to the medium and small HDL particles.

Another significant finding was that HDL cholesterol remained unchanged at the 120 and 180 minute postprandial times so the dietary effect occurred entirely as an increase in the particle numbers without adding cholesterol. At the time of this publication low HDL2b cholesterol was generally thought to be an indicator of cardiovascular risk, and there were few publications looking at increased risk associated with high or low HDL3 cholesterol. The conclusion of the study was that the high protein diet gave a less favorable medium and small HDL response compared to the high mono-unsaturated fat diet even though the high protein diet for medium and small HDL particles (HDL2a and HDL3 particles) showed a significant increase (p=0.01) over the fasting sample and the mono-unsaturated fat diet.

It is an object of the present invention to provide a method to detect cardiovascular disease.

It is an object of the present invention to provide a method to assess cardiovascular risk.

It is another object of the present invention to provide a method to reconcile both the inverse relationship and direct relationship of HDL3 particles to cardiovascular disease.

It is another object of the present invention to provide a method to reliably correlate HDL3 particles to cardiovascular disease.

It is another object of the present invention to provide a method to provide an assay of HDL3 to determine cardiovascular risk.

It is still another object of the present invention to provide a method to determine cardiovascular risk by detecting postprandial changes in HDL3.

It is still another object of the present invention to provide a method to determine cardiovascular risk by detecting postprandial changes in particle numbers for the lipoprotein subgroup HDL3.

These and other objects and advantages of the present invention will become apparent from a reading of the attached specification.

### BRIEF SUMMARY OF THE INVENTION

Embodiments of the present invention include a method for assessing a patient. The method comprises the steps of: drawing a first blood sample from a patient in a fasting condition; administering a liquid based protein meal to the patient; drawing a second blood sample from the patient after a time period of 60-120 minutes after the step of administering the meal; measuring HDL3 particles of the first blood sample so as to determine a fasting HDL3 particle amount; measuring HDL3 particles of the second blood sample so as to determine a postprandial HDL3 particle amount; comparing the fasting HDL3 particle amount and the postprandial HDL3 particle amount; and detecting cardiovascular risk based on the comparison.

In alternative embodiments of the invention, the fasting condition is comprised of at least eight hours of fasting completed by the patient before the step of drawing the first blood sample.

In alternative embodiments of the invention the liquid base protein meal is comprised of one half of a recommended dietary allowance of protein. For instance, the recommended dietary allowance can be 0.8 g of protein per kg of body weight per day. When the recommended dietary allowance is for a person with moderate physical activity, the recommended dietary allowance can be 0.6 g of protein per kg of body weight per day. Thus, in that embodiment, the liquid protein meal is comprised of 0.3 g of protein per kg of body weight of the patient. Generally, the liquid base protein meal is high protein, low carbohydrate, and low fat with known grams of protein per ounce.

In alternative embodiments of the invention, the time period is equal to a postprandial time period corresponding to a statistically significant increase in HDL particles of a protein meal over a mono unsaturated fat meal. The statistically significant increase in HDL particles is determined by protein, and the statistically significant increase in HDL particles is determined by an increase in HDL3 particles, not any HDL2 particles. In some examples, the time period ranges between 60-120 minutes. In particular embodiments, the time period is 90 minutes.

In alternative embodiments of the invention, the step of comparing is comprised of: subtracting the fasting HDL3-P amount from the postprandial HDL3-P amount so as to determine an HDL3-P activation index, and the step of detecting is comprised of determining a health profile corresponding to cardiovascular fitness and matching the HDL3-P activation index to the health profile. The health profile can be comprised of an increase in the HDL3-P activation index, equivalent HDL2b-P particle amounts in the fasting condition and in the postprandial condition, and equivalent HDL-C cholesterol amounts in the fasting condition and in the postprandial condition. The health profile is not just the HDL3-P activation index alone. In some embodiments, the increase in the HDL3-P activation index is between 0-100%. The HDL3-P is considered to be a primary lipoprotein increased for improved macrophage and cellular free cholesterol efflux.

The method further comprises making a treatment recommendation according to the step of matching. When a patient matches a health profile of an individual with a metabolic syndrome with high cardiovascular risk factors, the patient can be prescribed medications and activities to improve cardiovascular health. The patient can also be warned to avoid certain activities.

In alternative embodiments of the invention, the step of detecting is comprised of: determining an overall cardiovascular risk score for the patient; and correlating the HDL3-P activation index to the overall cardiovascular risk score. The HDL3-P activation index can be inversely related to the overall cardiovascular risk score. Thus, the method can further comprise making a treatment recommendation according to the step of correlating. The patient with a low HDL3-P activation and a high cardiovascular risk score can be prescribed medications and activities to improve cardiovascular health. The patient can also be warned to avoid certain activities.

### BRIEF DESCRIPTION OF THE SEVERAL VIEWS OF THE DRAWINGS

FIG. 1 is a schematic view of a table showing HDL subgroups.

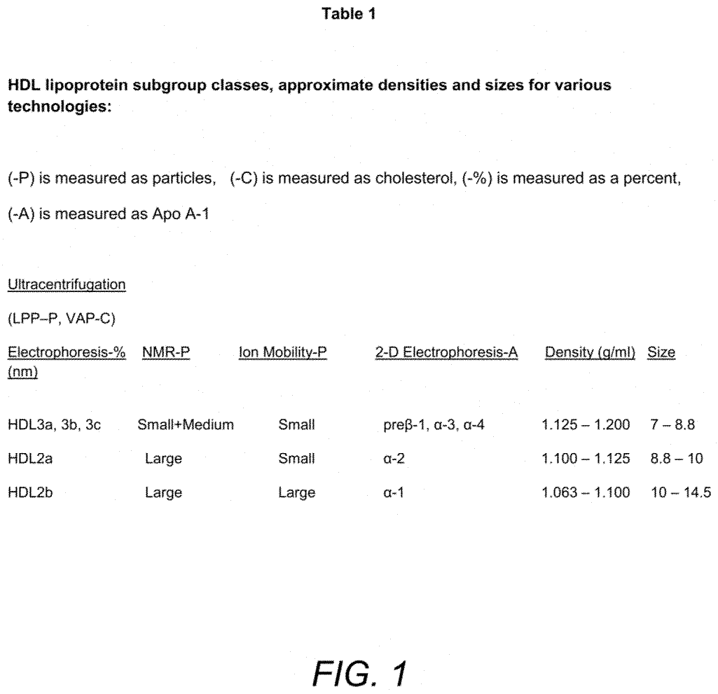

FIG. 2 is a schematic view of a table showing results of healthy individuals and individuals with a metabolic syndrome.

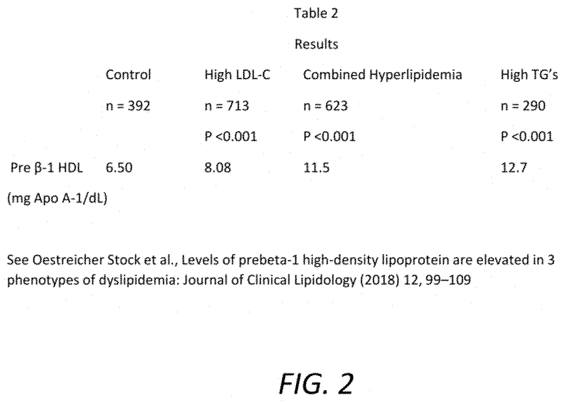

FIG. 3 is a schematic view of a graph illustration for determining the HDL3-P Activation Index, according to an embodiment of the method of the present invention.

FIG. 4 is a schematic view of another graph illustration for determining the HDL3-P Activation Index, according to an embodiment of the method of the present invention.

FIG. 5 is a schematic view of a table showing Postprandial HDL3-P Activation, according to an embodiment of the method of the present invention.

FIG. 6 is a schematic view of a graph illustration for a health profile of a healthy patient for the method according to the present invention.

FIG. 7 is a schematic view of a graph illustration for a health profile of a metabolic syndrome patient for the method according to the present invention.

FIG. 8 is a schematic view of a graph illustration for a health profile of a low fasting HDL3-P patient for the method according to the present invention.

FIG. 9 is a schematic view of a table showing results of the HDL3 Study, according to an embodiment of the method of the present invention.

FIG. 10 is a schematic view of Table 2, showing Pearson Correlation Coefficients.

### DETAILED DESCRIPTION OF THE INVENTION

Studies have shown that HDL2a particles and especially HDL2b particles are fully loaded with cholesterol and are near the end of the reverse cholesterol transport cycle for cholesterol delivered to the liver.

The HDL2b particle was traditionally thought to be the main indicator of cardiovascular risk since the HDL2b particle is the end result of HDL cholesterol loading. A low risk patient or healthy individual should have approximately 25 percent or more HDL particles as HDL2b particles. There is no doubt that low HDL2b particles, as in metabolic syndrome patients, indicates reduced functionality of HDL.

The cardiovascular risk assessment associated with HDL has recently added another dimension, HDL3 particles. There must be a balance between HDL2b and HDL3, since HDL3 is required as the precursor of HDL2b. Also, the ability of the body to produce more HDL3 particles for a different pathway of efficient reverse cholesterol transport is critical as determined in a number of studies. There are different functionalities of HDL3 that require consideration before any single measurement of HDL3 can be interpreted or relied upon as a risk factor.

For example, HDL3 can affect the understanding of HDL2b along the reverse cholesterol transport process. Low amounts of HDL2b particles, as in metabolic syndrome patients, indicate reduced functionality of HDL, but the reverse proposition that very high amounts of HDL2b particles indicate health and good HDL functionality may not necessarily be true. Instead, very high amounts of HDL2b may indicate an inability to complete the cycle of reverse cholesterol transport, that is, an inability to unload stored cholesterol to the liver. In that example, measurement of HDL3 would be a better indicator of cardiovascular risk and the potential of HDL to complete the reverse cholesterol transport process. HDL3 particles start the reverse cholesterol process from HDL3 to HDL2 to the liver and the direct reverse cholesterol process from HDL3 to the liver. Previous measurements of HDL2 as indicators of cardiovascular health did not account for HDL3, and the previous measurements of HDL3 were not interpreted correctly or ignored. In the prior art, a higher amount of fasting HDL3 was interpreted to indicate the inability to complete the reverse cholesterol transport process through HDL2. The prior art considered the result to be a less favorable medium that too much HDL3 meant that HDL2 was not being formed to complete the reverse cholesterol transport process.

Simply measuring HDL3 does not allow a reliable determination of cardiovascular risk. There are also existing methods to simply measure the amount of lipoproteins in a blood sample. The Lipoprotein Particle Profile (LPP) method for lipoprotein particle number measurement has been described in detail in the prior art, including U.S. Pat. No. 7,856,323, issued on Dec. 21, 2010 and WIPO publication WO 2016/049528 A1, published on Mar. 31, 2016. Other known measurements of HDL3 particle types may also be incorporated into the present invention. Known lipoprotein detection methods can already measure the subgroups of all lipoproteins and especially the HDL subgroups needed in the present invention which is aimed at the dietary functionality of HDL3.

The present invention addresses the need for a method to apply the step of measuring HDL3 particles in order to reliably determine cardiovascular risk.

The conventional step of measuring particles is in the fasting condition. Most lipids are measured in the fasting state to get a more accurate measure of triglycerides; however, the fasting state is not representative of the daily lipoprotein activity. People are in a non-fasting state most of the day, so the levels of lipoproteins and especially HDL subgroups, such as HDL3, are not the same as in the fasting patient. Therefore, the prior art measurements of HDL activity are biased views of the HDL particles for reverse cholesterol transport. Further complicating the assessment of lipoprotein activity is the fact that lipoprotein particles numbers can change in different ways from their cholesterol content. HDL-P for particles and HDL-C for cholesterol available to the HDL-P particles are different measurements. Generally, it has been observed that the lipoprotein particle numbers (HDL-P) in non-fasting patients are very similar to lipoprotein particle numbers (HDL-P) in fasting patients, except for Very Low Density Lipoproteins (VLDL) lipoproteins, where much of the triglycerides are stored. Non-fasting VLDL particles numbers show a shift from the smaller VLDL subgroup of VLDL3 to the larger particles of VLDL1 & 2. Other lipoprotein subgroups were not known to make much of an adjustment.

The prior art has previously addressed the steps of measuring HDL particles in both the fasting condition and the non-fasting condition. The results showed that a high protein (HP) meal compared to a high mono unsaturated fat (HMF) meal had no significant change in the cholesterol (HDL-C), but total particles (HDL-P) and the medium-small particles (HDL3+HDL2a particles of the HDL-P total) showed a significant increase (p=0.01). The prior art determined that the HP meal was a “less favorable medium” compared to the HMF meal, since the HDL-C cholesterol was unchanged and the larger HDL2b particles did not increase.

The significance of the increase in HDL-P particles overall due to the medium-small particles (HDL3+HDL2a) is largely unexplained in the prior art. As a side note in the prior art studies, the overall conclusion still supported established guidelines for increasing HDL-C cholesterol and the larger HDL2b particles. Having the increase in the HDL3 did not contradict the findings to increase HDL-C cholesterol and the larger HDL2b particles. There was no suggestion, motivation, or teaching that the prior art steps of measuring HDL particles in a fasting condition and measuring HDL particles in the postprandial condition can provide any other information, other than confirmation of known conclusions for dietary recommendations and cardiovascular health.

The present invention addresses the need for a method to apply the step of measuring HDL3 particles in a fasting condition and the step of measuring HDL3 particles in a postprandial condition in order to reliably determine cardiovascular risk.

Embodiments of the present invention include a method for assessing a patient for cardiovascular risk. The method comprises the steps of: drawing a first blood sample from a patient in a fasting condition; administering a liquid based protein meal to the patient; drawing a second blood sample from the patient after a time period of 60-120 minutes after the step of administering the meal; measuring HDL3-P of the first blood sample so as to determine a fasting HDL3-P amount; measuring HDL3-P of the second blood sample so as to determine a postprandial HDL3-P amount; comparing the fasting HDL3-P amount and the postprandial HDL3-P amount; and detecting cardiovascular risk based on the comparison.

According to embodiments of the step of drawing the first blood sample, the fasting condition is comprised of at least eight hours of fasting completed by the patient before the step of drawing the first blood sample. The eight hours without food or beverage intake, other than water, is a conventional standard for blood tests.

According to embodiments of the step of administering the liquid based protein meal, a specific dietary supplement is needed, similar to a suitable supplement for an oral glucose tolerance test. The meal of the present invention requires a high protein content and enough calories to generate an appropriate dietary response. Since the prior art determined the HDL3-P particle increases after the high protein meal, it was necessary to determine whether the HDL3-P particle increases related to the high protein content or the low fat content. In limited testing, it appeared that the addition of either fat or carbohydrate calories had little effect on the HDL3-P particle increases or HDL3-P activation. Furthermore, it was reasonable to select a high protein meal because the first step in the production of HDL3 is the formation of Apo A-1, a lipoprotein formed from the amino acid nutrients in a high protein drink. The Apo A-1 is combined with phospholipids to form a nacent pre-Beta HDL. Thus, embodiments of the present invention disclose a high protein drink as the meal to separate the fasting condition and the postprandial condition.

The relative “high protein meal or drink” required further standardization. In the present invention, the liquid base protein meal is comprised of one half of a recommended dietary allowance of protein. The Recommended Dietary Allowance (RDA) for a healthy adult is 0.8 grams of protein per kg of body weight per day. See Wu, G., Food Function, 2016 March; 7(3):1251-65. A person with moderate physical activity needs 1.3 grams of protein per kg of body weight per day or 0.6 grams per pound per day so a good estimate of a person's meal requirement would be one half of this dietary allowance or 0.3 grams of protein per pound of body weight. A “high” amount of protein is half of the total amount of protein recommended for the entire day in a single meal. Generally, the liquid base protein meal is high protein, low carbohydrate, and low fat with known grams of protein per ounce. A number of commercially available high protein liquid based meals are also considered for the liquid based protein meal of the present invention. Desirable liquid based meals are ones that have at least 40 grams of protein and minimal fat and carbohydrate calories.

According to embodiments of the step of drawing the second blood sample, the time period is equal to a postprandial time period corresponding to a statistically significant increase in HDL particles of a protein meal over a mono unsaturated fat meal. The prior art studies showed that between 120 minutes and 180 minutes, there was not a significant change in medium and small HDL-P, the HDL3-P particles. Thus, shorter postprandial times of 75, 90, and 100 minutes were tested, and the responses of the HDL3-P particles were not significantly different within the range of 60-120 minute time period. Since the present invention utilizes the high protein meal, the statistically significant increase in HDL particles should be determined by protein, not fat as previously discussed. Furthermore, the statistically significant increase in HDL particles is determined by an increase in HDL3 particles, not any HDL2 particles. In particular embodiments, the time period is 90 minutes, wherein the current assay protocol is to draw blood on a fasting patient and then have the patient drink a liquid based high protein meal and wait for 90 minutes before making a second blood draw. The timing of the second draw is flexible for at least 15 minutes after the 90 minutes waiting time since it was shown that there was no significant change in HDL3-P from 75 to 120 minutes in the prior art studies.

According to embodiments for the steps of measuring HDL3 of the first blood sample and measuring HDL3 of the second blood sample, the present invention relies on known particle measurement methods, including Ultracentrifugation as in the Lipoprotein Particle Profile (LPP (registered trademark)), Ion Mobility, 2-Dimensional electrophoresis, and NMR technology.

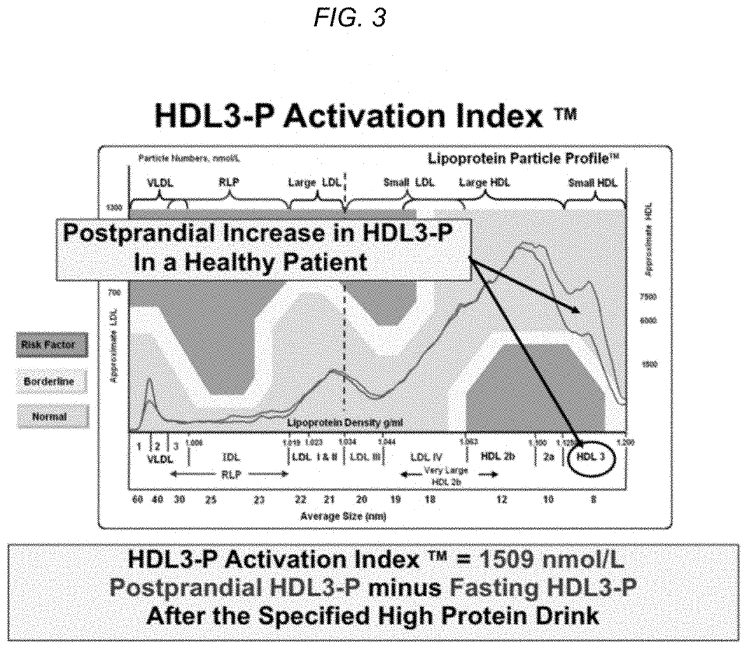

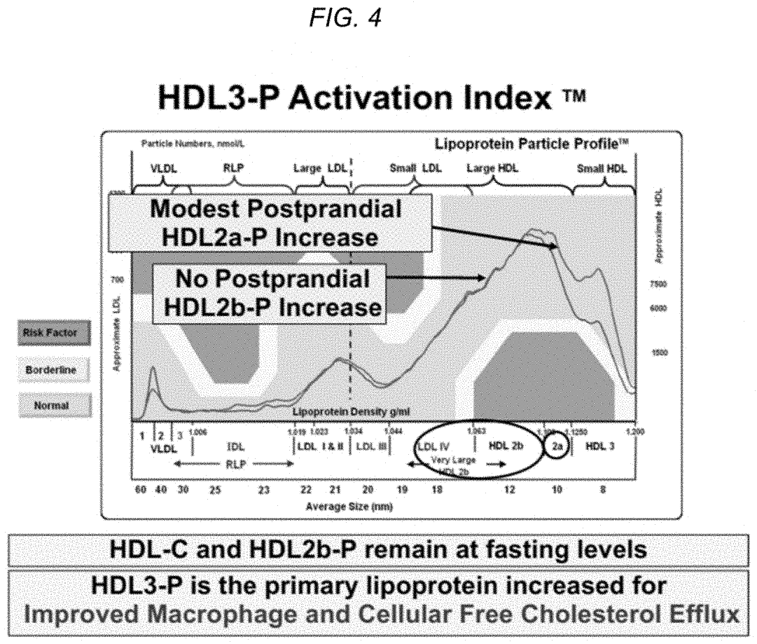

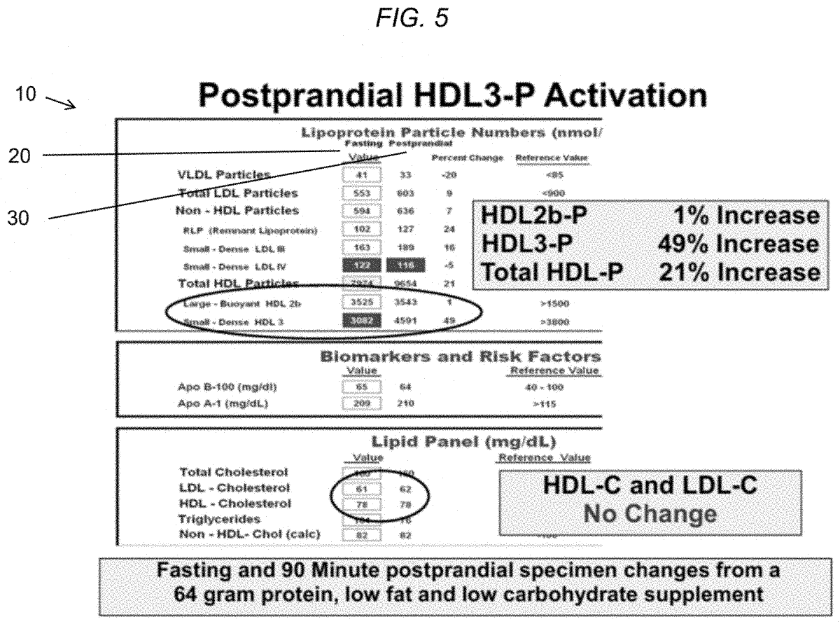

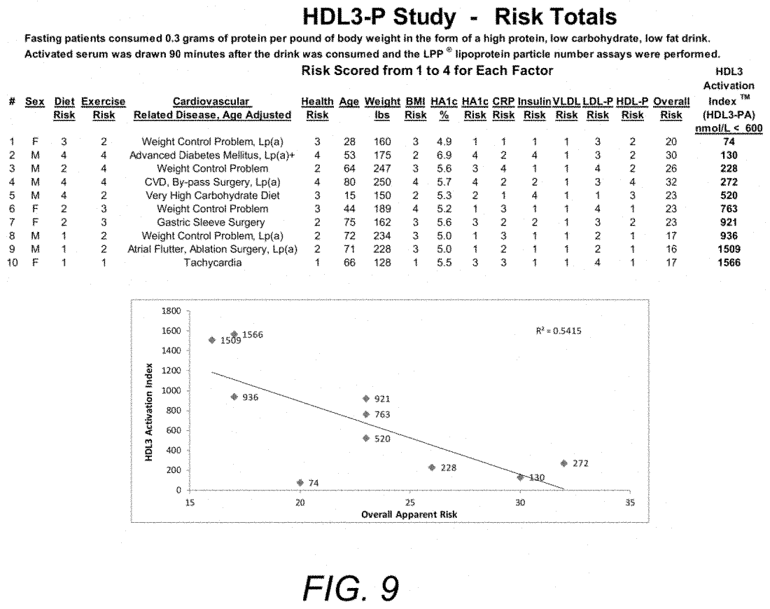

According to embodiments for the step of comparing, the present invention includes subtracting the fasting HDL3-P amount from the postprandial HDL3-P amount so as to determine an HDL3-P activation index, while the step of detecting is comprised of determining a health profile corresponding to cardiovascular fitness and matching the HDL3-P activation index to the health profile. FIG. 3 shows the determination of the HDL3-P activation index. FIGS. 4 and 5 show the determination of the health profile with factors besides the HDL3-P activation index, specifically, the modest HDL2a-P increase, the lack of HDL2b-P increase, and the equivalent HDL-C cholesterol amounts in the fasting condition and in the postprandial condition. The health profile can be comprised of an increase in the HDL3-P activation index, equivalent HDL2b-P particle amounts in the fasting condition and in the postprandial condition, and the equivalent HDL-C cholesterol amounts in the fasting condition and in the postprandial condition. The health profile is not just the HDL3-P activation index alone. In some embodiments, the increase in the HDL3-P activation index is between 0-100%. Based on the test results of FIG. 9, the increase in total HDL3-P corresponds to an increase in Total HDL-P of over 30%, and the lowest quartile for HDL3 activation index is less than 600 nmol/L, setting less than 600 nmol/L as a low HDL3 activation index. The HDL3-P is considered to be a primary lipoprotein increased for improved macrophage and cellular free cholesterol efflux.

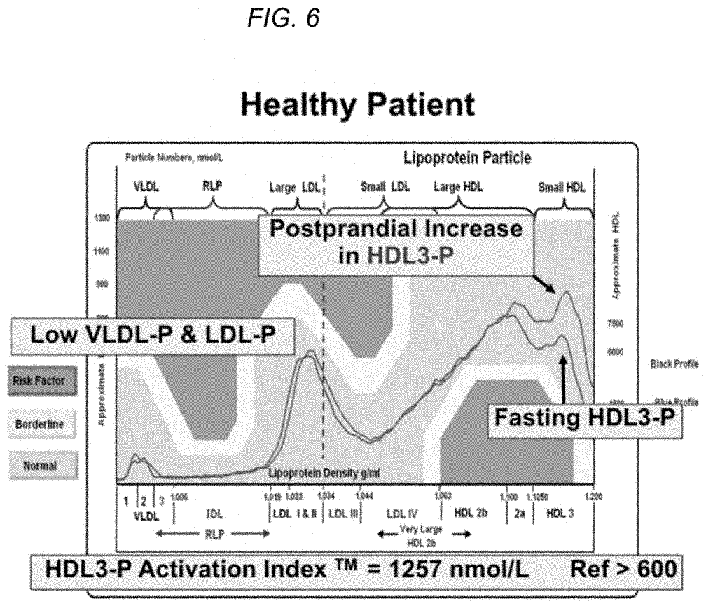

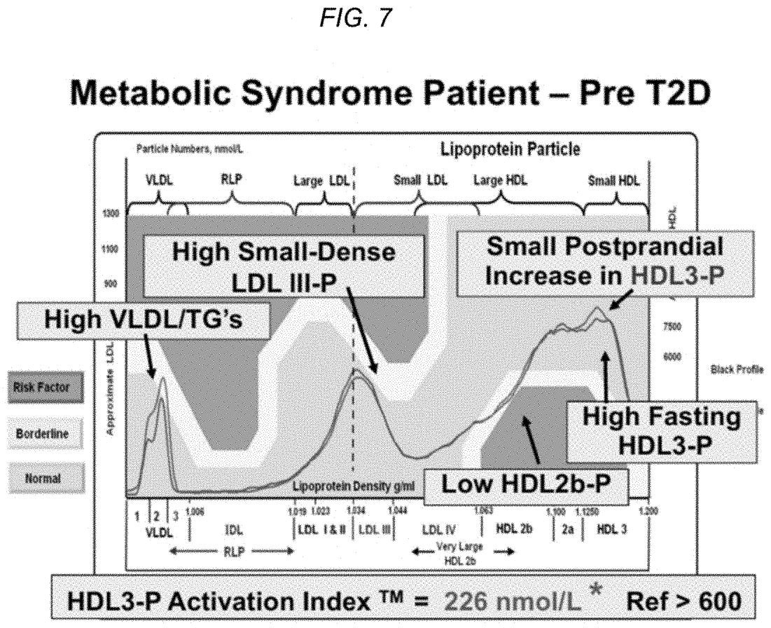

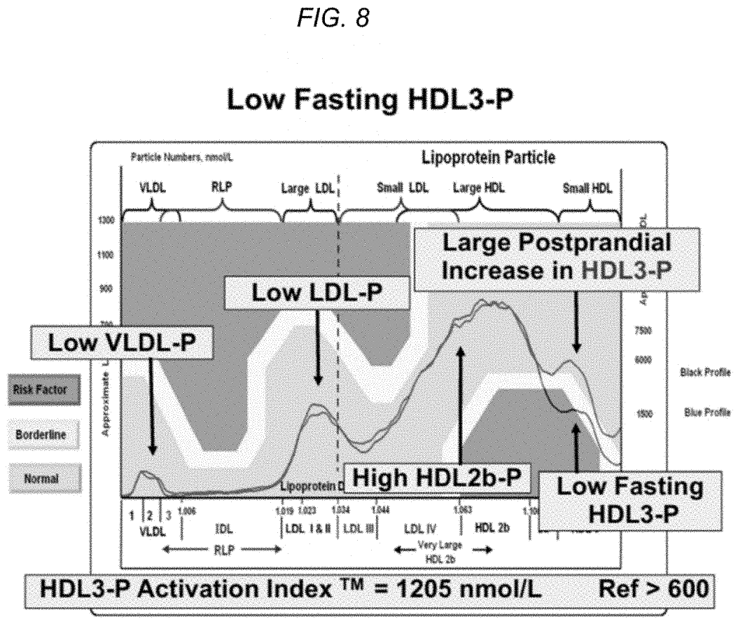

Embodiments of the method further comprise making a treatment recommendation according to the step of matching. When a patient matches a health profile of an individual with a metabolic syndrome with high cardiovascular risk factors, the patient can be prescribed medications and activities to improve cardiovascular health. The patient can also be warned to avoid certain activities. FIG. 6 shows a health profile of a healthy individual. FIG. 7 shows a health profile of a metabolic syndrome patient. FIG. 8 shows how the prior art can be reconciled with the present invention, wherein low amounts of fasting HDL3-P had previously different interpretations. In FIG. 8, the low fasting HDL3-P is not necessarily determinative of cardiovascular risk itself. The HDL3-P activation index remains high, so this individual is still able to react with HDL3-P pathways for direct reverse cholesterol transport.

According to alternate embodiments for the step of detecting is comprised of: determining an overall cardiovascular risk score for the patient; and correlating the HDL3-P activation index to the overall cardiovascular risk score. FIG. 9 shows determination of the overall cardiovascular risk score and the comparison to the HDL3-P activation index. The HDL3-P activation index can be inversely related to the overall cardiovascular risk score. Similar to other embodiments, the method can further comprise making a treatment recommendation according to the step of correlating. The patient with a low HDL3-P activation and a high cardiovascular risk score can be prescribed medications and activities to improve cardiovascular health. The patient can also be warned to avoid certain activities.

The present invention discloses a method to measure the full potential of the HDL3-P particles in a non-fasting environment, when there is a maximum effect on reverse cholesterol transport. The present invention more closely resembles the non-fasting state of daily life. A number of variables exist that need to be determined, such as composition of the dietary supplement and the optimal length of postprandial time for the measurement. However, the general prior art disclosures of postprandial effects determined by meal selection and time period tested after the meal fail to disclose the method of the present invention.

It is known that patients can vary dramatically in the HDL3-P increase due to dietary supplementation and with the length of postprandial time. Specifically, patients with high very buoyant HDL2b-P often have low fasting levels of HDL3-P that increases with a meal. Also, patients with low HDL2b-P often had high HDL3-P levels that did not change with a meal. FIG. 9 shows that some patients had a modest or no improvement in HDL3-P, where other patients showed a significant increase. Additional work is necessary to further all results of the present invention, but the evidence clearly supports a relationship between HDL3-P and cardiovascular risk. For example, it is not yet been proven that the percent (30%) or quantity (less than 600 nmol/L) as measured in nmol/L of HDL3 particle numbers is directly and causally related to cardiovascular health or if only the final response is more predictive of reverse cholesterol transport and risk. The HDL3 levels measured in fasting patients in the prior art were inconclusive regarding cardiovascular risk with little guidance to resolve the discrepancies between studies. The present invention presents a method to reconcile the current state of the prior art.

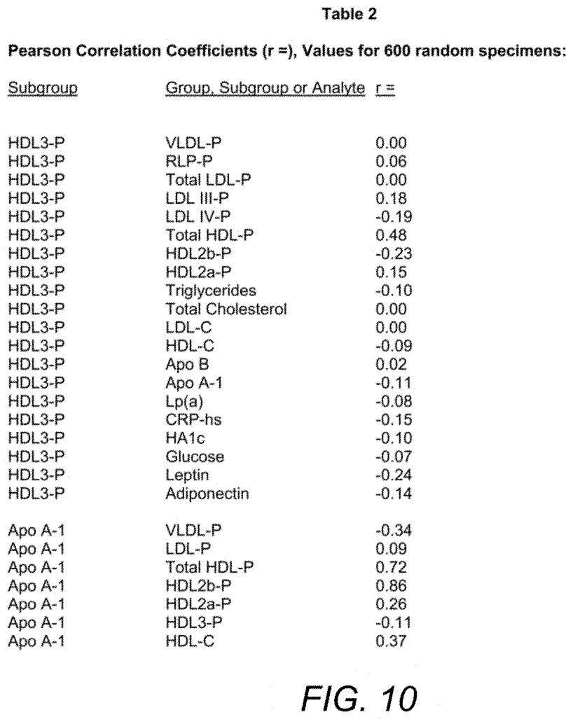

An in-house correlation study of 600 random patients in FIG. 10 indicated that HDL3-P is truly an independent marker which agrees with previous work. The largest lipoprotein correlation was with Total HDL-P which is understandable since HDL3-P it is a component of HDL-P. HDL3-P was poorly or negatively correlated with HDL2a-P and HDL2b-P which emphasizes the importance of measuring only HDL-3-P and not including HDL2a-P or medium size HDL. Interestingly, negative weak correlations exist between high sensitivity C-reactive protein (CRP-hs), an inflammation marker and fat metabolism markers of Leptin and Adiponectin since these are all associated with cardiovascular risk. Also, Apo A-1 correlations were examined since Apo A-1 is the indicator in 2-D electrophoresis measurements and the main protein on HDL. A high correlation of Apo A-1 with HDL2b of r=0.86 was found and a very low correlation of r=0.26 for HDL2a. For HDL3 a correlation of r=−0.11 was determined which is probably due to the fact that one or two Apo A-1 molecules are present on HDL3. This observation makes the quantitative measurements of HDL subgroups, using Apo A-1 as the indicator, subject to large errors.

The measurement of the dietary response of lipoproteins and more specifically HDL3-P can be a useful metric, not only to determine cardiovascular risk but also in the testing of various therapies to stimulate HDL production. A number of studies have been done using Cholesterol Ester Transfer Protein (CETP) inhibitors to improve HDL for risk reduction. These approaches have failed in clinical trials but this may be due to targeting the production of HDL2b-C and not the production of HDL3-P. Until now a method to study HDL3-P without interference from HDL2a-P has not been developed. Additionally, most studies looked at HDL cholesterol rather than particle numbers so the benefit of increasing HDL3-P was not noticed. The dietary influence on HDL3-P was not studied in the CETP trials so the true level of HDL activation was unknown.

The HDL Activation Assay:

This purpose of the present invention is as an assay to measure the postprandial levels of HDL3-P to determine cardiovascular risk and HDL3-P activation. Some prior art studies have shown that fasting low levels of HDL3-C or HDL3-P are associated with higher risk and some studies show that high levels of HDL3-C or HDL3-P are associated with cardiovascular disease. Since HDL3 is needed for indirect reverse cholesterol transport, low levels of HDL3 could impair this process. Low levels of HDL3 in the fasting condition is not necessarily bad and associated with cardiovascular disease (See FIG. 8). Most of the day a person is in a non-fasting state, so it is logical that the postprandial values of HDL3-P are important. Since total HDL-C does not increase in the postprandial state it cannot be used to measure this potential. From previous work it was shown that a meal, especially a high protein meal will stimulate the body to produce HDL3-P particles that are nearly free of cholesterol. This is the activation of HDL3-P and believed to be important in cardiovascular risk.

The results of a fasting patient of the present invention show a significant postprandial increase in HDL3-P, that the HDL3-P particles must have performed their function by being converted to HDL2b-P or returning to the liver as HDL3 particles to complete the reverse cholesterol transport cycle. This direct reverse cholesterol transport cycle is also an example of healthy functioning HDL. In metabolic syndrome patients, where small postprandial increases in HDL3-P are observed and low fasting HDL2b-P is present that doesn't change with a meal, the reverse cholesterol transport cycle is dysfunctional. The small postprandial increases represent a disease state that can be detected by the assay of the present invention and is not accurately measured by a fasting HDL3 assay alone.

Metabolic syndrome patients usually have high fasting HDL3-P but this is different type of HDL3-P that has been formed by the action of CETP and hepatic lipases in the presence of elevated triacylglycerols. This enzymatic activity reduces the size of HDL2's to that of an HDL3. For clarity, this type of HDL3 is HDL3X-P. It appears that HDL3X-P is not recognized by hepatic SR-B1 (scavenger receptor B1) for functional reverse cholesterol transport so in spite of a reasonable total HDL particle number, metabolic syndrome patients actually have very little HDL2b-P and non-functional HDL3X-P.

The methods of measuring HDL3 are known. The profiles of HDL particles are composed from the integrated intensity of 20 Gaussian profiles at specific densities that define each lipoprotein group and subgroup. HDL-P is composed of two Gaussians for HDL2b-P positioned at a density of 1.063-1.100 g/ml, one for HDL2a-P positioned at a density of 1.100-1.125 g/ml and two for HDL3-P positioned at a density of 1.125-1.200 g/ml. The result for HDL3-P was not previously reported separately but rather as a component of total HDL-P since there was no clinical guidance for this result.

Accurate individual measurement of small, HDL3-P particle numbers has not been reported previously without the influence of mid-sized HDL-P or HDL2a-P. As shown in the lipoprotein particle profile result (FIG. 4), there is a clear separation of HDL2a-P and HDL3-P and these results have a very low Pearson Correlation Coefficient of HDL3-P to HDL2a-P of r=0.15 (FIG. 10, Table 2). The current Lipoprotein Particle Profile now reports HDL3-P as a separate result.

The present invention is a method to measure the difference between fasting and postprandial lipoprotein particle number values to determine cardiovascular risk. On the lipoprotein particle profile report up to four measurements can be presented to compare results and perform calculations related to those results. The present invention is focused on the cardiovascular risk association from the activation of lipoprotein subgroup HDL3-P. In FIG. 8, patients with average to higher fasting HDL3-P values often had modest percentage increases in HDL3-P and this appeared to be associated with metabolic syndrome type profiles or higher cardiovascular risk. In one embodiment, a reference value was determined for a small number of activated specimens representing patients with various levels of cardiovascular disease. This value was determined to be approximately 600 nmol/L.

The Cardiovascular Risk and HDL3-P Activation of the assay from the method of the present invention can be evaluated based on results from:

1. The final postprandial increased HDL3-P value.

2. The increase in HDL3-P between the fasting and postprandial results.

3. The changes that result between fasting and postprandial results for all lipoprotein subgroups.

FIG. 9 shows cardiovascular and health risk scoring from assay results for a study from a variant population. HDL3-P activation was shown to change from 0% to 100% depending on the patient. In other words, the up to a 100% increase in previously unknown reverse cholesterol transport potential. The increase also corresponds to an increase in total HDL-P of over 30%. More importantly, the study shows an indication of direct correlation with both apparent health and apparent cardiovascular risk of the patients. Poor HDL3-P activation appears to be associated pre-diabetic or diabetic patients, existing atherosclerosis, obesity and poor physical condition and strong family history of atherosclerosis. These cardiovascular risk conditions can be treated if identified early. Since HDL3-P appears to be the primary component in reverse cholesterol transport and the delivery of anti-oxidants it may very well be the single most important lipoprotein yet in the fasting state its actual potential in unknown. The HDL3-P activation assay has the ability to reveal the true patient cardiovascular risk.

More detailed studies based on the best correlation between the above three metrics and other lipid and advanced lipoprotein results, other analytes—many of which are listed in FIG. 10, additional medical diagnostics, height, weight, blood pressure, exercise, diet, possible disease states, medication, vitamins and supplements.

The foregoing disclosure and description of the invention is illustrative and explanatory thereof. Various changes in the details of the described method can be made without departing from the true spirit of the invention.

I claim:

1. A method (10) for assessing a patient, the method comprising the steps of:
drawing a first blood sample from a patient in a fasting condition;
administering a liquid based protein meal to the patient;
drawing a second blood sample from the patient after a time period of 60-120 minutes after the step of administering the meal;
measuring HDL3-P of the first blood sample so as to determine a fasting HDL3-P amount (20);
measuring HDL3-P of the second blood sample so as to determine a postprandial HDL3-P amount (30);
comparing said fasting HDL3-P amount and said postprandial HDL3-P amount; and
detecting cardiovascular risk based on step of comparing.

2. The method for assessing, according to claim 1, wherein said fasting condition is comprised of at least eight hours of fasting completed by said patient before the step of drawing said first blood sample.

3. The method for assessing, according to claim 1, wherein said liquid base protein meal is comprised of one half of a recommended dietary allowance of protein.

4. The method for assessing, according to claim 3, wherein said recommended dietary allowance is 0.8 g of protein per kg of body weight per day.

5. The method for assessing, according to claim 3, wherein said recommended dietary allowance is for a person with moderate physical activity.

6. The method for assessing, according to claim 5, wherein said recommended dietary allowance for said person with moderate physical activity is 0.6 g of protein per kg of body weight per day

7. The method for assessing, according to claim 6, wherein said liquid protein meal is comprised of 0.3 g of protein per kg of body weight of said patient.

8. The method for assessing, according to claim 1, wherein said liquid base protein meal is high protein, low carbohydrate, low fat with known grams of protein per ounce.

9. The method for assessing, according to claim 1, wherein said time period is 90 minutes.

10. The method for assessing, according to claim 1, wherein said time period is equal to a postprandial time period corresponding to a statistically significant increase in HDL particles from a protein meal over a mono unsaturated fat meal.

11. The method for assessing, according to claim 10, wherein said statistically significant increase in HDL particles is determined by protein.

12. The method for assessing, according to claim 10, wherein said statistically significant increase in HDL particles is determined by an increase in HDL3 particles.

13. The method for assessing, according to claim 1,
wherein the step of comparing is comprised of:
subtracting said fasting HDL3-P amount from said postprandial HDL3-P amount so as to determine an HDL3-P activation index; and
wherein said step of detecting is comprised of:
determining a health profile corresponding to cardiovascular fitness; and
matching said HDL3-P activation index to said health profile.

14. The method for assessing, according to claim 13, wherein said health profile is comprised of an increase in said HDL3-P activation index, equivalent HDL2b-P particle amounts in said fasting condition and in said postprandial condition, and equivalent HDL-C cholesterol amounts in said fasting condition and in said postprandial condition.

15. The method for assessing, according to claim 14, wherein said increase in said HDL3-P activation index is between 0-100%.

16. The method for assessing, according to claim 14, further comprising the step of:
making a treatment recommendation according to the step of matching.

17. The method for assessing, according to claim 1, wherein said step of detecting is comprised of:
determining an overall cardiovascular risk score for said patient; and
correlating said HDL3-P activation index to said overall cardiovascular risk score.

18. The method for assessing, according to claim 17, wherein said HDL3-P activation index is inversely related to said overall cardiovascular risk score.

19. The method for assessing, according to claim 17, further comprising the step of:
making a treatment recommendation according to the step of correlating.
